(19) United States
(12) Patent Application Publication
(10) Pub. No.: US 2006/0134611 A1
(43) Pub. Date: Jun. 22, 2006

(54) Condom device for detecting STD's

(76) Inventor: Derrick Dwayne Danzy, Tallahassee, FL (US)

Correspondence Address: Raynarldo K. Whitty;The Pittman Law Group, 528 E. Park Avenue, Tallahassee, FL, 32301, US

(21) Appl. No.: 10/851,736

(22) Filed: May 24, 2004

(51) Int. Cl. C12Q 1/70

(52) U.S. Cl. 435/5

(57) ABSTRACT

A condom device for detecting STD's comprising an outer layer having open pores positioned along the surface of the outer layer. The condom device includes an inner layer positioned opposite the outer layer. An intermediate layer is positioned between the outer layer and the inner layer. The intermediate layer receives bodily fluid for testing of pre-determined STD's. An indicator is positioned against at least one of the outer layer and the inner layer, wherein the indicator changes color to notify the wearer of a positive STD test result.

### CROSS REFERENCE TO RELATED APPLICATIONS

(Not Applicable)

### STATEMENT REGARDING FEDERALLY SPONSORED RESEARCH OR DEVELOPMENT

(Not Applicable)

### BACKGROUND OF INVENTION

1. Technical Field

The present invention relates generally to a condom device. More particularly, The invention relates to a condom device with enhanced physical properties for the detection of sexually transmitted disease.

2. Description of the Related Art

Condoms are used for several purposes ranging from the prevention of sexually transmitted disease (STD) to the prevention of pregnancy. Regardless of the purpose, a condom must maintain durability to serve the particular purpose for which it is being utilized. The vast majority of condoms have been primarily constructed to prevent the transmission of STD's via the use of prophylactic material to prevent the exchange of bodily fluid between consenting partners. However, most condom devices do not serve the purpose of alerting a user of the presence of a particular STD's. For example, condoms are constructed primarily to protect the user from the transmission of a STD by preventing the exchange of bodily fluid. However, in the event of breakage, the wearer is exposed to bodily fluids and consequently is not provided with any notification of the presence of any types of STD after breakage. Additionally, the likelihood of a condom being damaged via a rupture or breakage increases during usage, thereby increasing the chances of possible exposure.

Several attempts have been made to develop an apparatus that combines the benefits of STD detection and protection. U.S. Pat. No. 4,910,803 to Cukier discloses an apparatus directed to a composite material adaptable to form bodily fluid detection apparel such as gloves and condoms. Moreover, the apparatus alerts the wearer that the protective apparel is breached and thereby no longer serving its protective function. While the Cukier apparatus can be adequate solution for the basic purpose and function for which it has been specifically designed. When condom breakage occurs, the Cukier design is deficient with respect to its failure to provide notice to the wearer of the detection of a particular sexually transmitted disease, and is not the most efficient and convenient structure for prevention of sexually transmitted diseases.

Other devices in the prior art are contain tamper or damage indicating members but are also deficient in consequence of being able to only indicate breakage and failure to indicate the presence of a specific sexually transmitted disease. For example, U.S. Pat. No. 6,370,694 B1 to Michelson describes a condom having a leak detection mechanism for the purpose of protecting the wearer from disease. The Michelson device contains material that changes color to violet upon contact with bodily fluids to indicate a breach in the outer layer. However, the Michelson device is not the most efficient to provide for the detection and notification of the presence of any sexually transmitted diseases. The Michelson device is deficient because it only notifies the wearer in the event of a condom break occurring and does not provide any information to the wearer concerning the presence of a particular life threatening sexually transmitted diseases, such as Acquired Immune Deficiency Syndrome. In consequence, often users are not aware that they have been exposed to a particular STD regardless of an actual break occurring, and even in situations wear breakage has occurred the wearer is not provided with any notification of the presence of a particular STD, which could be beneficial regarding medical treatment.

Condom devices can be ineffective for the purpose of prevention of the detection of sexually transmitted diseases because many of the devices available in the prior art fail to adequate notified the user of the presence of a condom break and subsequent notification of the presence of a particular sexually transmitted disease. In consequence, many individuals experience condom breakage and are not informed of their possible exposure to life threatening sexually transmitted diseases.

Condom devices that are utilized to provide a means to detect condom breaks should provide an indicator means to detect the presence of a sexually transmitted disease before breakage occurs, where oftentimes it is too late. Many STD's provide no symptoms or signs for several months, and condom experience failure rates of approximately 17%. Accordingly, there exists a need for an improved condom device that can be utilized to detect and notify a user of the presence of a particular sexually transmitted disease, while also providing increased strength and durability.

### SUMMARY OF INVENTION

The present invention relates to a condom device that provides enhanced performance characteristics for the detection of sexually transmitted disease (STD).

According to the invention, a condom device for detecting sexually transmitted diseases can comprise an outer layer and an inner layer. The outer layer of the condom device can include a plurality of open spaces located on the surface of said outer layer. The device also can include an intermediate layer. The intermediate layer can be positioned against at least one of the outer layer and the inner layer of the condom device and receive bodily fluid for testing of pre-determined STD's. The intermediate level can also include an indicator means to notify the wearer of a positive test result.

The outer layer and inner layer provide protection against fluid as it contacts the surface of the condom. In one embodiment of the present invention, the plurality of open pores located on the outer layer can be utilized to transport bodily fluids. Bodily fluids coming into contact with the outer layer of the condom can be transferred from the outer layer to the intermediate layer. The open spaces can be arranged in a periodic relationship across the surface of the condom to allow a minimal amount of bodily fluid to pass through said openings in order to test the fluid for the presence of sexually transmitted diseases.

The plurality of open pores can also extend from the outer layer to the inner layer of the condom device. The open spaces allow fluid to pass intermittently from the outer layer to the intermediate layer. Incremental amounts of bodily fluid pass through the pores. The intermediate layer is located between the outer layer and inner layer. The intermediate layer can be utilized to test for the presence of sexually transmitted diseases.

The intermediate layer can also include an indicator means to notify the wearer that a sexually transmitted disease has been detected. Upon the detection of a particular sexually transmitted disease the indicator means can notify the user of such detection via a color change or other suitable means. For example, bodily fluid can pass through the pores of the outer layer into the intermediate layer. Once the fluid has entered the intermediate layer it is tested for a particular sexually transmitted disease, such as Acquired Immune Deficiency Syndrome (AIDS).

In one embodiment of the present invention, the condom device can include an indicator means. The indicator means can provide notice of a positive test result via a color change utilizing a particular color to indicate a positive result for a corresponding disease. For instance, upon detection of a particular disease, the indicator means can change color, such as violet or photoluminescent, to indicate a positive test result. The indicator means can also detect a damage or breakage in the outer or inner layer of the condom device. Upon the detection of a particular STD or breakage, an indicator means can be utilized to notify the wearer. The color change can be based on any adequate testing means such as measuring ph changes, pressure gradients, toxic measurements or other suitable method. The indicator means can be utilized to notify the wearer of the presence of STD's, such as AIDS, herpes, chlamydia or other testable STD.

Several variations of material can be utilized as material for the condom device. The outer layer can be formed using a membrane having a geometry that allows the open spaces to facilitate very small amounts of fluids into the intermediate space. One embodiment of the invention can include a membrane that allows certain viruses, bacteria, or other pathogens to pass through the outer layer to be tested for disease. In this embodiment, the plurality of open spaces can be defined by sidewalls that decreases in cross sectional area as the open spaces extend from the outer layer toward the intermediate and inner layer allowing only incremental amounts of bodily fluid to pass.

The intermediate layer, which is defined by the outer layer and inner layer can serve various functions. In additional to operating as a testing area for STD's, this area can also be utilized to disinfected bodily fluids through release of chemicals. For example, in other embodiments of the invention the open spaces can be arranged in a periodic configuration or any other suitable configuration. The release of chemical disinfectants can be utilized to destroy any bacteria that may carry infections. Additionally, the open spaces can be filled with alternative lubricants and spermicides, thereby providing enhanced physical properties to the condom device.

The condom device can be composed of a variety of materials including, but not limited latex or non-latex condoms. Examples of non-latex materials suitable for use include polyurethane. In one embodiment of the present invention, the outer layer can be composed of a semi-permeable or permeable membrane, such as animal skin. Types of animal skin can include lamb skin or sheep skin. Semi-permeable material can allow certain bodily fluids to pass through the outer layer into the intermediate layer. Once in the intermediate layer, the inner layer can be composed of a non-permeable material, such as latex, to prevent any further transport of bodily fluids.

Another aspect of the present invention is to provide information to the wearer regarding exposure to potentially life threatening diseases. The indicator means can alert the wearer of possible exposure to pre-determined viruses or bacteria associated with a particular disease. Upon receipt of this information the wearer can decrease any further exposure to any deadly pathogens and use information to provide to a medical examiner and provide a long term treatment plan. Similar to early pregnancy test that provide notice of impending pregnancy, the present invention can provide the wearer with notice of exposure and/or the presence of STD's in a timely fashion. As many STD's are skin exposed around the area of a condom, this device can further provide the wearer with useful information to enable the wearer to make an informed decision regarding further exposure to such pathogens.

### BRIEF DESCRIPTION OF THE DRAWINGS

The invention may be better understood by referring to the following description taken in conjunction with the accompanying drawings, it being understood, however, that the invention is not limited to the precise arrangements and instrumentalities shown.

FIG. 1 shows a perspective view of a condom device for the detection of sexually transmitted disease (STD);

FIG. 2 shows a top-cross sectional view of a condom device having separate layers;

FIG. 3 shows a top view of the surface of the outer layer of the condom device having a plurality of open pores;

FIG. 4 shows a side cross sectional view of the condom device having separate layers; and

FIG. 5 shows a perspective view of the condom device having an indicator displaying a color change on the outer layer.

### DETAILED DESCRIPTION OF THE PREFERRED EMBODIMENTS

The present invention provides a condom device 10 with enhanced physical properties. The invention provides a condom device 10 comprised of various layers of material to provide enhanced physical capabilities and increased protection against sexual transmitted disease (STD).

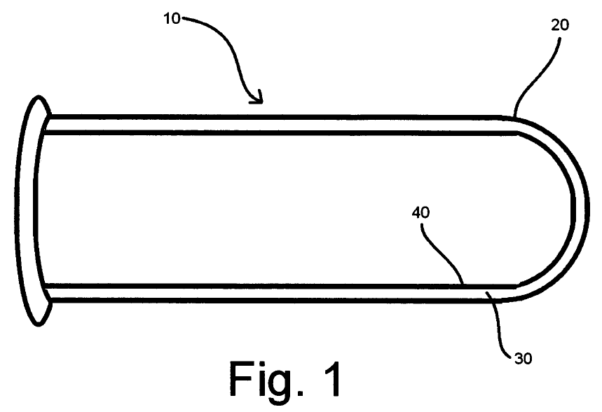

FIG. 1 depicts an embodiment of a condom device 10 for the detection of STD's. The condom device 10 comprises an outer layer 20 having open pores 50 positioned along the surface of the outer layer 20. The condom device also includes an inner layer 40 positioned opposite the outer layer 20. An intermediate layer 30 can be positioned between the outer layer 20 and the inner layer 40. The intermediate layer 30 receives bodily fluid for testing of pre-determined STD's. An indicator 60 can be positioned against at least one of the outer layer 20 and the inner layer 40. The indicator 60 notifies the wearer of a positive result for a particular STD.

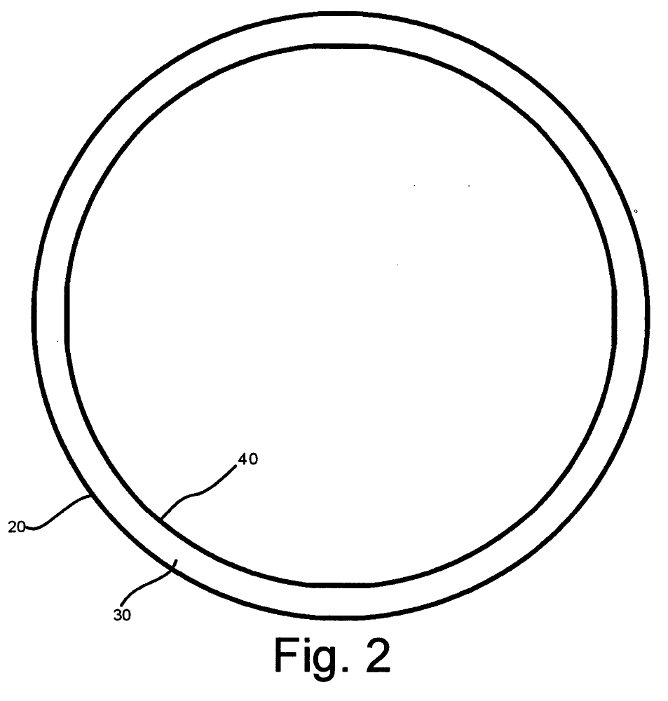

An embodiment of the present invention, as illustrated in FIG. 2, can include an outer layer 20 that facilitates the passage of bodily fluid from outside of the outer layer 20 to the intermediate layer 30 of the condom device 10. The passage of fluid can be transported to the inner layer 40 for further testing. Only incremental amounts of liquid are allowed to pass through the outer layer 20 of the condom. The outer layer 20 provides a barrier from pathogenic elements coming into contact with the device 10. Further, the outer layer 20 functions as the first stage of STD testing whereby certain bodily fluids can be blocked and prevented from passage via material integrated with the outer layer 20.

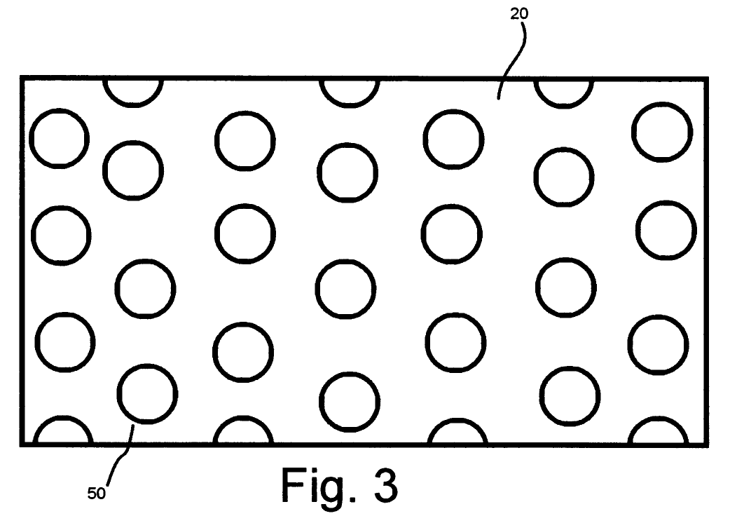

FIG. 3 shows the open pores 50 located on the surface of the outer layer 20 of the condom device 10. The plurality of open pores 50 located on the surface of the outer layer 20 can extend from the outer layer 20 to the intermediate layer 30. Additionally, the pores 50 can extend to the surface of the inner layer 40. The pores 50 located on the outer layer 20 can vary in size and shape. Pores 50 can also be defined by sidewalls that decrease in cross sectional area as the open pores 50 extend from the outer layer 20 to the inner layer 40. In an embodiment of the present invention illustrated in FIG. 3, the pores 50 can be arranged in a periodic positioning across the surface of the outer layer 20 and include a circular shape. However, the size and shape of the pores 50 is not limited to a circle, and can include other shapes, such as a rectangle, square and octagon. The sizes of the pores 50 can vary in size to further block the passage of fluid containing a particular pathogenic bacteria and viruses while allowing the passage of others. For example, a pore size can be adjusted to allow the passage of bacteria consisted with that of gonorrhea during testing for this particular STD, while blocking fluid containing the HIV virus. Conversely, pore size can be adjusted to allow the passage of viruses consistent with that of HIV during testing for this particular STD, while blocking fluid containing the gonorrhea bacteria. Alternatively, the pore size can be adjusted to allow the passage of fluid to the intermediate layer 30 to provide an overall test for multiple STD's, such as HIV and gonorrhea.

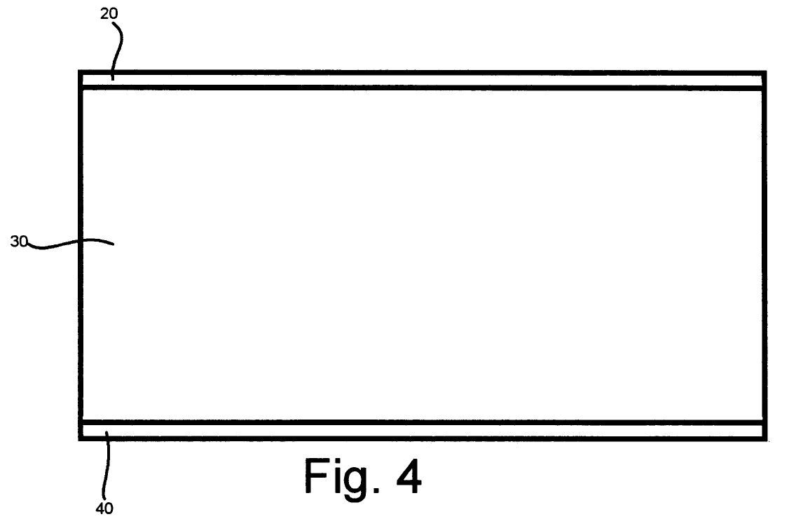

As shown in FIG. 4, the intermediate layer 30 of the condom device 10 can receive varying forms of bodily fluid for testing. Body fluids can include semen, vaginal fluid, perspiration, saliva or other forms of bodily fluid that may carry disease. The intermediate layer 30 can also include reactants and other agents to test any received fluid for particular STD's. In one embodiment of the present invention, the intermediate layer 30 can be used as a testing site for sexually transmitted diseases and the presence of other bacteria and viruses that facilitate the transport of sexually transmitted disease. The device 10 can test for sexually transmitted disease such as Human Immunodeficiency Virus 1 and 2 (HIV-1 & HIV 2), Acquired Immune Deficiency Syndrome (AIDS), Chlamydia and various other disease. For instance, in one embodiment of the present invention the intermediate layer 30 can include reactants that test for HIV infection after fluid is received in intermediate layer 30. HIV infection usually is diagnosed by tests for antibodies against HIV-1 and HIV-2 (HIV-1/2). Antibody testing can begin with a sensitive screening test (e.g., the enzyme immunoassay [EIA]). Reactive screening tests must be confirmed by supplemental test (e.g., the Western blot [WB]) or an immunofluorescence assay (IFA). If confirmed by a supplemental test, a positive antibody test result-indicates that a person is infected with HIV and is capable of transmitting the virus to others. HIV antibody is detectable in at least 95% of patients within 3 months after infection. Although a negative antibody test result usually indicates that a person is not infected, antibody tests cannot exclude recent infection. Once a positive result is detected. The indicator 60 can indicate a positive result by a corresponding color change in the outer layer 20.

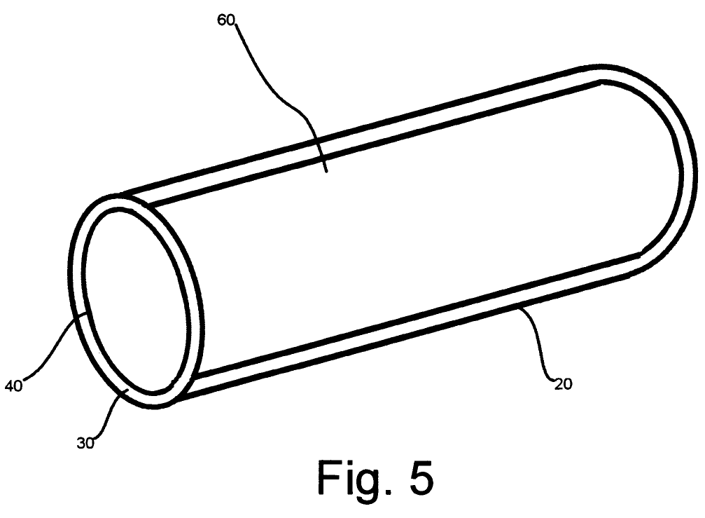

As shown in FIG. 5, the condom device 10 can include an indicator 60. The indicator 60 can alert the wearer via a color change or other suitable means detectable in the outer layer 20 of the condom device 10. The color change can be indicated by a violet color or by a color that is easily identified in the dark, such as a phosphorous (glow in the dark) material. FIG. 5 further illustrates an indicator 60 alerting the wearer through the use of a color change on the outer layer 20 of the condom device 10. A phosphorous material or photoluminescent material is useful as it provides the user of a positive test result that is highly recognizable under conditions of dim lighting. For example, if the condom worn by the wearer begins to glowing-the-dark during usage while the lights are off, the wearer can recognize the alert and will be informed that of the potential exposure of an STD. Additionally, the condom device 10 can include an indicator 60 that alerts the wearer of possible exposure to an STD using a violet color change, which is highly visible in lighted areas.

In one embodiment of the present invention, the device 10 can test for other infections such as vaginal infections. Bodily fluid containing vaginal infection is usually characterized by a vaginal discharge or vulvar itching and irritation. Also, a vaginal odor may be present. The three diseases most frequently associated with vaginal discharge are trichomoniasis (caused by T. vaginalis), bacterial vaginosis (caused by a replacement of the normal vaginal flora by an overgrowth of anaerobic microorganisms, mycoplasmas, and Gardnerella vaginalis), and candidiasis (usually caused by Candida albicans). Additionally, C. trachomatis (Chlamydia) or N. gonorrhoeae (Gononorrhea) can sometimes cause vaginal discharge. Although vulvovaginal candidiasis and bacterial vaginosis are not usually transmitted sexually, these bacteria can be tested in the intermediate layer 30 of the condom device 10. During testing for bacterial infections occurring in the intermediate layer 30, the bodily fluid transported from the outer layer 20 can be tested by pH and microscopic testing of the discharge collected in the intermediate layer 30. For example, the pH of the bodily fluid collected from vaginal secretions can be determined with a result of an elevated pH (>4.5) typical of BV or trichomoniasis. Bodily fluid collected in the intermediate layer 30 can be examined by diluting the collected fluid with one to two drops of 0.9% normal saline solution while in the intermediate layer 30. Additionally, the intermediate layer 30 can include 10% potassium hydroxide (KOH) solution. Once the bodily fluid reactant with such reactants in the intermediate layer 30 an amine odor can be an indication of bacterial vaginosis (BV). The motile T. vaginalis or the clue cells of BV usually are identified easily using saline specimens. The yeast or pseudohyphae of Candida species are more easily identified using a KOH specimen. A positive test result for vulvovaginal candidiasis, trichomoniasis and bacterial vaginosis in the intermediate layer 30 can also be identified with a color change in the intermediate layer 30. An indication of a positive result utilizing the device 10 should prompt the wearer of possible infection and assist in efforts to reduce the risk behavior that could lead to possible transmission to the wearer. Additionally, the wearer is provided with information that allows the wearer to make an informed decision whether to engage in any high risk activities.

The condom device 10 can also be utilized to test for the presence of Human Papillomavirus Infection (HPV), commonly recognized as genital warts. HPV infections are usually asymptomatic, unrecognized, or subclinical. However, visible genital warts usually are caused by HPV types 6 or 11. Other HPV types in the anogenital region (e.g., types 16, 18, 31, 33, and 35) have been strongly associated with cervical neoplasia. Detection of genital warts can be found in the intermediate layer 30 utilizing the presence of HPV nucleic acid tests in the intermediate layer 30. In addition, reactants can be included in the intermediate layer 30 to test for antibodies causing common STD's, such as Hepatitis B, Herpes Simplex Virus 2, Syphilis, Epididymis, and Crab Lice (Condyloma Acuminata).

The condom device 10 can be composed of latex or other suitable material to provide protection from pathogenic bodily fluids. Suitable material utilized to compose the outer layer 20 can include and is not limited to polyurethane, polyethylene, vinyl, polypropylene and polyvinychloride. Additionally, in one embodiment of the invention, the outer layer 20 can be composed of a semi-permeable material selected from the group of lamb skin and sheep skin. This type of material allows fluid to flow from the outer layer 20 at an increased level due to its slight permeability. The inner layer 40 can also be composed from materials such as polyurethane, polyethylene, vinyl, polypropylene and polyvinychloride.

It will of course be understood that the invention is not limited to the specific details described herein, which are given by way of example only, and that various modifications and alterations are possible within the scope of the invention as defined in the appended claims. For example, the condom device 10 can include variations in the outer layer 20, configurations of open pores 50, and testing reactants for various STD's in the intermediate layer 30, and remain within the scope of the invention.

What is claimed:

1. A condom device comprising:
an outer layer, said outer layer having open pores positioned along said surface of outer layer;
an inner layer positioned opposite said outer layer; and
an intermediate layer positioned between said outer layer and said inner layer, wherein said intermediate layer receives bodily fluid for testing of pre-determined sexually transmitted disease; and
an indicator positioned against at least one said outer layer and said inner layer; wherein said indicator changes color to notify the wearer of a positive STD test result.

2. The condom device according to claim 1, wherein said outer layer facilitates the transport of incremental amounts of fluid across the surface of said outer layer toward said intermediate layer.

3. The condom device according to claim 1, wherein said intermediate layer includes a reactant to test for the presence of a sexually transmitted disease.

4. The condom device of claim 3, wherein said reactant includes agents to test for antibodies of a group of sexually transmitted diseases consisting of human immunodeficiency virus, acquired immune deficiency syndrome, gonorrhea and chlamydia.

5. The condom device of claim 3, wherein said reactant includes agents to test for antibodies of a group of sexually transmitted diseases consisting of herpes, vaginosis, trichomoniasis and candidiasis.

6. The condom device of claim 3, wherein said reactant includes agents to test for antibodies of a group of sexually transmitted diseases consisting of syphilis, condyloma acuminatum, hepatis B and epididymis.

7. The condom device according to claim 1, wherein said plurality of open pores transport bodily fluid from the surface of the outer layer to the intermediate layer.

8. The condom device according to claim 6, wherein said open pores filter pathogenic material from bodily fluid transported through said pores of outer layer.

9. The condom device according to claim 1, wherein said plurality of open pores therein extend from said outer layer toward said intermediate layer.

10. The condom device according to claim 1, wherein said plurality of open pores therein extend from said outer layer toward said inner layer.

11. The condom device according to claim 1, wherein said plurality of open pores formed by sidewalls decrease in cross section area as said open pores extend from said outer layer toward said inner layer.

12. The condom device according to claim 1, wherein said plurality of open pores are arranged in a periodic relationship across said surface of said outer layer.

13. The condom device according to claim 1, wherein said outer layer is composed of a material chosen from the group of materials consisting of at least one of latex, polyurethane and polyethylene.

14. The condom device according to claim 1, wherein said outer layer is composed of a material chosen from the group of materials consisting of at least one of vinyl, polypropylene and polyvinychloride.

15. The condom device according to claim 1, wherein said outer layer is composed of a semi-permeable material chosen from the group consisting of lamb skin and sheep skin.

16. The condom device according to claim 1, wherein said inner layer is composed of a material chosen from the group of materials consisting of at least one of latex, polyurethane and polyethylene.

17. The condom device according to claim 1, wherein said inner layer is composed of a material chosen from the group of materials consisting of at least one of vinyl, polypropylene and polyvinychloride.

18. The condom device according to claim 1, wherein said indicator changes to a violet color to indicate a positive test result for a sexually transmitted disease.

19. The condom device according to claim 1, wherein said indicator changes to a photoluminsecent color to indicate a positive test result.
